(19) United States
(12) Patent Application Publication
(10) Pub. No.: US 2007/0168038 A1
(43) Pub. Date: Jul. 19, 2007

(54) Materials, devices and methods for treating multiple spinal regions including the interbody region

(75) Inventor: Hai H. Trieu, Cordova, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/331,701

(22) Filed: Jan. 13, 2006

(51) Int. Cl. A61F 2/44

(52) U.S. Cl. 623/17.15; 623/17.16

(57) ABSTRACT

A method of treating a spinal condition includes placing disc replacement material in an intervertebral disc, thereby altering a loading characteristic of an adjacent vertebrae, and supporting the adjacent vertebrae by applying a bone filling material to either a superior or inferior vertebral body adjacent to the intervertebral disc. The disc placement material may also be configured to augment the intervertebral disc.

### BACKGROUND

The present application relates to the following applications, all of which are filed concurrently herewith, assigned to the same assignee, and are hereby incorporated by reference.

| | Attorney | |
|---|---|---|
| Title | Docket No. | Inventor(s) |
| Materials, Devices, and Methods for | P22656.00 | Hai H. Trieu |
| Treating Multiple Spinal Regions | 31132.377 | |
| Including The Anterior Region | | |
| Materials, Devices, and Methods for | P22578.00 | Hai H. Trieu |
| Treating Multiple Spinal Regions | 31132.376 | |
| Including The Posterior and Spinous | | |
| Process Regions | | |
| Materials, Devices, and Methods for | P22681.00 | Hai H. Trieu |
| Treating Multiple Spinal Regions | 31132.379 | |
| Including Vertebral Body and | | |
| Endplate Regions | | |
| Use Of A Posterior Dynamic | P22397.00 | Aure Bruneau et al. |
| Stabilization System With An | 31132.420 | |
| Interdiscal Device | | |

Disease, degradation, and trauma of the spine can lead to various conditions that require treatment to maintain, stabilize, or reconstruct the vertebral column. As the standard of care in spine treatment begins to move from arthrodesis to arthroplasty, preserving motion and limiting further degradation in a spinal joint or in a series of spinal joints becomes increasingly more complex. To date, standard treatments of the vertebral column have not adequately addressed the need for multiple devices, systems, and procedures to treat joint degradation. Likewise, current techniques do not adequately address the impact that a single treatment or arthroplasty system may have on the adjacent bone, soft tissue, or joint behavior.

### SUMMARY

The present disclosure describes materials, devices, and methods for treating multiple spinal regions including the interbody region. In one embodiment, a method of treating a spinal condition includes placing disc replacement material in an intervertebral disc, thereby altering a loading characteristic of an adjacent vertebrae, and supporting the adjacent vertebrae by applying a bone filling material to either a superior or inferior vertebral body adjacent to the intervertebral disc. In some embodiments, the disc placement material is configured to augment the intervertebral disc.

In another embodiment, a method of treating a spinal condition includes placing an augmentation material in an intervertebral disc, thereby altering a characteristic of a first vertebrae, and supporting the adjacent vertebrae by replacing at least a portion of a second, adjacent vertebral with an artificial vertebral body.

In another embodiment, a method of treating a spinal condition includes placing a disc replacement system in an intervertebral disc space between a pair of vertebrae and supporting the disc replacement system to maintain disc height by connecting an anterior system between the pair of vertebrae.

In another embodiment, a method of treating a spinal condition includes placing a disc replacement system in an intervertebral disc space and supporting the disc replacement system to maintain disc height by connecting an anterior system between a pair of vertebrae. The method further includes reinforcing at least one of the pair of vertebrae to support the disc replacement system by treating at least one of the pair of vertebrae with a vertebral body treatment material. In some embodiments, the method further includes reinforcing an endplate of at least one of the pair of vertebrae to support the disc replacement system by treating the endplate with an endplate treatment material.

In another embodiment, a method of treating a spinal condition includes placing a disc replacement system in an intervertebral disc space between a pair of vertebrae and reinforcing an endplate of at least one of the pair of vertebrae to support the disc replacement system by treating the endplate with an endplate treatment material.

In another embodiment, a method of treating a spinal condition includes placing a disc replacement system in an intervertebral disc space between a pair of vertebrae and reinforcing an endplate of at least one of the pair of vertebrae to support the disc replacement system by treating the endplate with an endplate treatment material.

Additional methods, devices, and systems are described below in the following description and the attached drawings.

### BRIEF DESCRIPTION OF THE DRAWINGS

FIG. 1 is a sagittal view of a section of a vertebral column.

FIG. 2 is a superior view of a vertebral body depicted in FIG. 1.

FIGS. 3-13 are sagittal views of a section of a vertebral column having multiple region treatments.

### DETAILED DESCRIPTION

The present disclosure relates generally to vertebral reconstructive devices, and more particularly, to systems and procedures for treating multiple spinal conditions. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

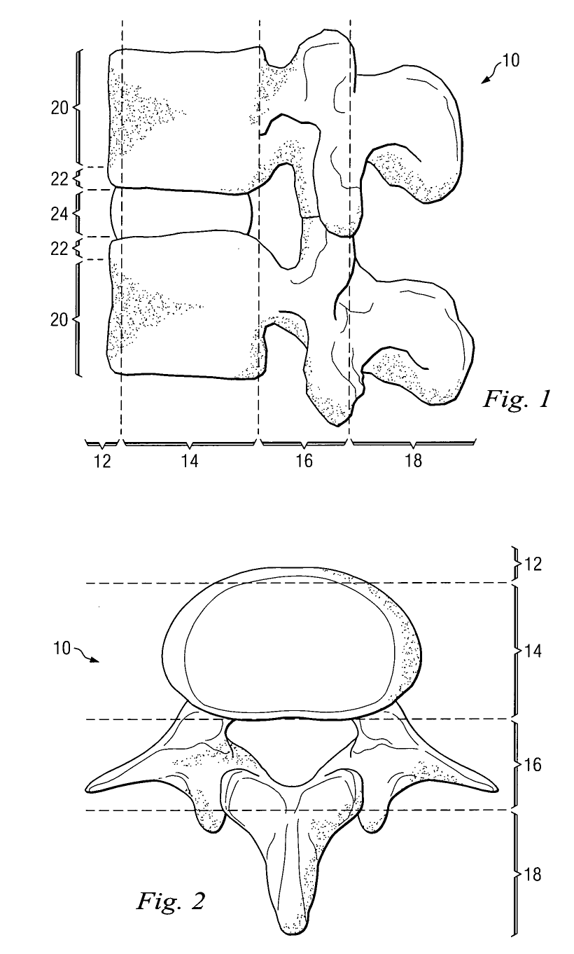

Referring first to FIGS. 1 and 2, the reference numeral 10 refers to a vertebral joint section or a motion segment of a vertebral column. The joint section 10 may be considered as having several regions extending from anterior to posterior. These regions include an anterior region 12, an anterior column region 14, a posterior region 16, and a spinous process region 18. The anterior column region 14 may be further considered to have several regions extending longitudinally along the vertebral column. These regions include a vertebral body region 20, an endplate region 22, and an interbody or disc space region 24.

Disc degeneration may lead to disc collapse or loss of disc height, resulting in pain or neurodeficit. Similarly, degeneration of the facet joints may lead to pain or neurodeficit. When treating one degenerated area of the vertebral joint, the impact of the treatment on the surrounding regions should be considered. For example, inappropriate restoration of disc height to only a posterior portion of the interbody space may result in hyperkyphosis with loss of height in the anterior interbody area and placement of the anterior annulus in compression. Likewise, in appropriate restoration of disc height to only an anterior portion of the interbody space may result in hyperlordosis with loss of posterior disc height and compression of the posterior annulus and facet joints.

Treatment, stabilization, and/or reconstruction of the vertebral joint section 10 may be diagnosed and carried out in a systematic manner depending upon the conditions and material or systems available for treatment. To achieve an improved clinical outcome and a stable result, multiple regions of the vertebral column may be treated.

Anterior

Anterior or anterolateral systems and devices for treating anterior region 12 may include synthetic or natural tissue based prostheses for replacing or supplementing the anterior longitudinal ligament (ALL). Alternatively, anterior or anterolateral systems may include anterior bone fixation plates for the cervical, thoracic, or lumbar vertebral regions. Such plates may include those offered by or developed by Medtronic, Inc. of Minneapolis, Minn. under brand names such as the ATLANTIS plate, PREMIER plate, ZEPHIR plate, MYSTIC plate, PYRAMID plate, or DYNALOK CLASSIC plate, CD HORIZON ECLIPSE. In still another alternative, anterior or anterolateral systems may be made of flexible materials such as woven or braided textile based devices, elastomer-based devices, or polymeric composite-based devices that connect with two or more vertebrae. In still another alternative, the anterior or anterolateral systems may include annulus repair or replacement devices for the anterior portion of the annulus. Some anterior systems may be bioresorbable or partially resorbable.

The anterior or anterolateral devices may connected to two or more vertebral bodies or vertebral endplates through the use of any connection mechanism such as bone screws, staples, sutures, or adhesives. The anterior or anterolateral systems may be loaded in compression or tension depending upon the patient's indication or the performance of other implanted systems or treatments. For example, an anterior plate may be installed in tension to counteract disc or facet degeneration in more posterior regions of the vertebral joint.

The anterior or anterolateral systems may be formed from a rigid material or configuration such as a titanium or stainless steel plate. Alternatively, systems may be formed of less rigid or more flexible materials such as polyaryletherketone (PAEK)-based materials, which includes polyetheretherketone (PEEK), polyetherketoneketone (PEKK), PEEK-carbon composite, polyetherimide, polyimide, polysulfone, polyethylene, polyester, polylactide, copolymers of poly L-lactide and poly D-lactide, polyorthoester, tyrosine polycarbonate, polypolyurethane, silicone, polyolefin rubber, etc. The systems may be formed of inelastic material, such as braided tethers or woven fabric of polyester or polyethylene, or of elastic material, such as rubber banding or plates, sheets, rods, or tubing made of silicone or polyurethane.

Interbody

The disc space may require treatment due to disc collapse or loss of disc height due to degeneration, disease, or trauma. Disc space or intervertebral body devices and systems for treating region 24 may include rigid fusion devices such as those offered by or developed by Medtronic, Inc. of Minneapolis, Minn. under brand names such as INTERFIX cage, INTERFIX RP cage, LT cage, CORNERSTONE spacer, TELAMON spacer, MDII and MDIII threaded bone dowels, PRECISION GRAFT and PERIMETER ring spacers, etc. Alternatively, interbody devices may include prosthetic motion preserving discs such as those offered by or developed by Medtronic, Inc. under brand names such as MAVERICK, BRYAN, PRESTIGE, or PRESTIGE LP. Single articulating surface motion preserving discs may be disclosed more fully in U.S. Pat. Nos. 6,740,118; 6,113,637; or 6,540,785 which are incorporated by reference herein. Double articulating surface motion preserving discs may be disclosed more fully in U.S. Pat. Nos. 5,674,296; 6,156,067; or 5,865,846 which are incorporated by reference herein. In still another alternative, motion preserving interbody devices may extend posteriorly from the interbody space and include features for providing posterior motion. These types of bridged systems may be disclosed in U.S. Pub. Pat. App. Nos. 2005/0171610; 2005/0171609; 2005/0171608; 2005/0154467; 2005/0154466; 2005/0154465; 2005/0154464; 2005/0154461 which are incorporated by reference herein. In still another alternative, a spherical, ellipsoidal or similarly shaped disc replacement device may be installed in the interbody space. Such devices may include the SATELLITE system offered by or developed by Medtronic, Inc. This type of device may be described in detail, for example, in U.S. Pat. No. 6,478,822 which is incorporated by reference herein. In still another alternative, a disc replacement device may be an elastically deformable device comprising a resilient or an elastomeric material such as silicone, polyurethane, polyolefin rubber or a resilient polymer, and/or may comprise a mechanical spring component.

Alternatively, interbody motion preserving devices may include nucleus replacement implants that work in conjunction with all or portions of the natural annulus. Such nucleus replacement implants may include those offered by or developed by Medtronic, Inc under a brand name such as NAUTILUS or offered by or developed by Raymedica, Inc. of Minneapolis, Minn. under brand names such as PDN-SOLO® and PDN-SOLO XL™. These types of nucleus replacement implants may be described in detail in, for example, U.S. Pat. Nos. 6,620,196 and 5,674,295 which are incorporated by reference herein. Injectable nucleus replacement material including a polymer based system such as DASCOR™ by Disc Dynamics of Eden Prairie, Minn. or a protein polymer system such as NuCore™ Injectable Nucleus by Spine Wave, Inc. of Shelton, Conn. may be alternatives for preserving interbody motion. Other acceptable alternative injectable or insertable disc augmentation biomaterials may be natural or synthetic and may include injectable and in situ curable polyurethane or an in situ curable poly vinyl alcohol compound. Injectable silicone or collagen may also be used to restore disc height and/or preserve joint motion. Injected collagen may be autogenic, allogenic, or synthetic and may be crosslinkable. Injectable materials may be used alone or together with an inflatable container implanted within the interbody space.

The interbody systems may be loaded in compression or tension depending upon the patient's indication or the performance of other implanted systems or treatments. These interbody systems may provide a desired level of intervertebral disc space distraction the depending upon the patient's indication. For example, an interbody device or system may be sized or filled to balance posterior interspinous distraction provided by an interspinous device.

Posterior

Posterior region systems for treating region 16 may extend along the posterior or posterolateral side of the vertebral column and may span one or more vertebral joints. Posterior systems may be used with intact anatomy or in situations in which one or more facet, the spinous process, or even the entire lamina have been resected. Examples of posterior region systems may include rigid fixation systems such as hook, rod, and screw systems which are offered by or developed by Medtronic, Inc. of Minneapolis, Minn. under brands such as CD HORIZON, CD HORIZON SEXTANT, CD HORIZON M8, CD HORIZON LEGACY, CD HORIZON ANTARES, COLORADO 2, EQUATION, VERTEX, TSRH, and TSRH-3D. Semi-rigid or flexible systems may also be used and may include systems offered by or developed by Medtronic, Inc. under brand names such as FLEXTANT or AGILE or offered by or developed by Zimmer, Inc. of Warsaw, Ind. such as the Dynesys® Dynamic Stabilization System. These types of flexible systems may be disclosed, for example, in U.S. Pat. Pub. Nos. 2005/0171540 and 2005/0131405. These particular systems may replace or supplement natural facet joints and may attach to the posterior features of adjacent vertebrae using bone screws. Additional systems may include Archus Othopedics, Inc.'s TOTAL FACET ARTHROPLASTY SYSTEM (TFAS™) or similar devices performing facet functions

Alternatively, dampener systems such as those described in U.S. Pat. Nos. 5,375,823; 5,540,688; 5,480,401 or U.S. Pat. App. Pub. Nos. 2003/0055427 and 2004/0116927, each of which is incorporated by reference herein. Additionally, rod and screw systems that use flexible PEEK rods may be chosen. In another alternative, posterior systems may be made of flexible materials such as woven or braided textile based devices that connect with two or more vertebrae. These flexible materials may be formed of natural graft material or synthetic alternatives. In still another embodiment, the posterior region systems may include annulus repair or replacement devices for the posterior portion of the annulus.

The posterior region systems and devices may connected to two or more vertebral bodies or vertebral endplates through the use of any connection mechanism such as bone screws, staples, sutures, or adhesives. The systems and devices may be loaded in compression or tension depending upon the patient's indication or the performance of other implanted systems or treatments. For example, a flexible device attached to adjacent vertebrae with bone screws may be installed in tension to balance disc degeneration or subsidence of an interbody prosthesis.

The posterior region systems may be formed from rigid materials such as a titanium or stainless steel. Alternatively, systems may be formed of less rigid or more flexible materials such as polyaryletherketone (PAEK)-based materials, which includes polyetheretherketone (PEEK), polyetherketoneketone (PEKK), PEEK-carbon composite, etc., polyetherimide, polyimide, polysulfone, polyethylene, polyester, polylactide, copolymers of poly L-lactide and poly D-lactide, polyorthoester, tyronsine polycarbonate, polypolyurethane, silicone, etc. The systems may be formed of inelastic material, such as braided tethers or woven fabric of polyester or polyethylene, or of elastic material, such as rubber banding or plates, sheets, rods, or tubing made of silicone or polyurethane. The systems may be formed of composite material including one or more materials listed above.

Spinous Process

Spinous process systems for treating region 18 may extend between adjacent spinous processes and/or extend around or through adjacent spinous processes. As one example, spinous process systems may include rigid interspinous process systems such as the Spire Plate system offered by or developed by Medtronic, Inc. of Minneapolis, Minn. or the X-Stop system offered by or developed by St. Francis Medical Technologies of Alameda, Calif. Such systems may be disclosed in U.S. Published App. No. 2003/0216736 or in U.S. Pat. Nos. 5,836,948; 5,860,977; or 5,876,404 which are incorporated by reference herein. Spinous process systems may also include semi-rigid spacer systems having flexible interspinous process sections and flexible ligaments or tethers for attaching around or through spinous processes. Such devices may include the DIAM system offered by or developed by Medtronic, Inc. or the Wallis system offered by or developed by Abbott Laboratories of Abbott Park, Ill. Semi-rigid spacer systems may be disclosed in greater detail in U.S. Pat. Nos. 6.626,944 and 6,761,720 which are incorporated by reference herein. Alternatively, semi-rigid spacer systems may have rigid interspinous process sections formed of materials such as titanium but incorporating flexible ligament or tethering devices that permit a limited amount of flexion-extension motion at the vertebral joint.

In still another alternative, spinous process systems may include artificial ligaments for connecting two or more spinous processes. In another alternative, interspinous process systems may be made of flexible materials such as woven or braided textile based tethers that connect with two or more vertebrae. Elastic or rubber-like materials may also be used in the interspinous process region. Depending upon the system chosen, the spinous process systems may be installed through open surgical procedures, minimally invasive procedures, injection, or other methods known in the art. These systems and devices may be loaded in compression or tension depending upon the patient's indication or the performance of other implanted systems or treatments.

Vertebral Body

Vertebral bodies may become damaged due to compressive trauma fractures or osteoporosis. The vertebral body region 20 may be treated to strengthen diseased or traumatized bone, reinforce bone adjacent to prosthetic implants, or repair bone loss caused by implantation or revision of prosthetic systems. One or more vertebral bodies may be treated with injectable or implantable biocompatible materials that can be placed into cancellous or cortical bone. The material may be allowed to solidify to provide structural support and reinforcement. Examples of suitable biocompatible materials may include bone cements such as those made from polymethylmethacrylate (PMMA), calcium phosphate, hyrdroxyapatite-tricalcium phosphate (HA-TCP) compounds, bioactive glasses, polymerizable matrix comprising a bisphenol-A dimethacrylate, or CORTOSS™ by Orthovita of Malvern, Pa. (generically referred to as a thermoset cortical bone void filler). Calcium sulfate bone void fillers and other filling materials or combinations of filling materials may also be used. Bone void fillers or bone cements may be treated with biological additives such as demineralized bone matrix, collagen, gelatin, polysaccharide, hyaluronic acid, keratin, albumin, fibrin, cells and/or growth factors. Additionally or alternatively, bone void fillers or bone cements may be mixed with inorganic particles such as hydroxyapatite, fluorapatite, oxyapatite, wollastonite, anorthite, calcium fluoride, agrellite, devitrite, canasite, phlogopite, monetite, brushite, octocalcium phosphate, whitlockite, tetracalcium phosphate, cordierite, berlinite or mixtures thereof .

Other osteoinductive, osteoconductive, or carrier materials that may be injected, extruded, inserted, or deposited into vertebral bone include collagen, fibrin, albumin, karatin, silk, elastin, demineralized bone matrix, or particulate bone. Various bone growth promoting biologic materials may also be used including mysenchymal stem cells, hormones, growth factors such as transforming growth factor beta (TGFb) proteins, bone morphogenic proteins (including BMP and BMP2), or platelet derived growth factors. Examples of such materials that can be injected into vertebral bodies are disclosed in U.S. Pub. No. 2005/0267577, which is hereby incorporated by reference.

The above mentioned bone fillers may be used alone such as in vertebroplasty procedures that inject bone cement directly into the interstitial spaces in cancellous bone. Alternatively, the above mentioned bone fillers and treatments may be used with void creation devices such as balloon expansion systems offered by or developed by Kyphon, Inc. of Glendale, Calif. examples of such systems are disclosed in U.S. Pub. Nos. 2004/0102774 and 20040133280 and U.S. Pat. Nos. 4,969,888 and 5,108,404, all of which are incorporated by reference herein. Other void creation systems that utilize expandable cages or displacement systems may also be used for vertebral body repair. Such systems may be disclosed in U.S. Published Pat. App. No. 2004/0153064 and 2005/0182417 and are incorporated by reference herein. In still another alternative, vertebral body replacement devices or corpectomy devices may be used to replace an entire vertebrae or series of vertebrae. Such corpectomy systems may be of the type disclosed, for example, in U.S. Pat. Nos. 5,702,453; 5,776,197; 5,5776,198; or 6,344,057 which are incorporated by reference herein.

Endplate

Endplates may become fractured, damaged, or collapsed as a result of degeneration, disease, or trauma. Even relatively healthy endplates may need reinforcement due to procedures that affect surrounding regions. The endplate region 22 of vertebral body 20 may be replaced, reinforced or otherwise treated to strengthen the area in preparation for further procedures or to repair damage caused by interbody procedures such as disc replacement surgery. Endplate supplementation systems may use rigid or flexible devices such as metal plates with spikes or other attachment mechanisms to anchor the plates to existing bony tissue. Alternatively, vertebral endplates may be treated with injectable or implantable biocompatible materials that can be placed into cancellous or cortical bone. The material may be allowed to solidify to provide structural support and reinforcement. Examples of suitable biocompatible materials may include bone cements such as those made from polymethylmethacrylate (PMMA), calcium phosphate, hyrdroxyapatite-tricalcium phosphate (HA-TCP) compounds, bioactive glasses, polymerizable matrix comprises a bisphenol-A dimethacrylate, or thermoset cortical bone void filler . Calcium sulfate bone void fillers and other filling materials or combinations of filling materials may also be used. These implant materials may be treated with biological additives such as demineralized bone matrix, collagen, gelatin, polysaccharide, hyaluronic acid, keratin, albumin, fibrin, cells and/or growth factors. Additionally or alternatively, the implant materials may be mixed with inorganic particles such as hydroxyapatite, fluorapatite, oxyapatite, Wollastonite, anorthite, calcium fluoride, agrellite, devitrite, canasite, phlogopite, monetite, brushite, octocalcium phosphate, Whitlockite, tetracalcium phosphate, cordierite, Berlinite or mixtures thereof.

Other osteoinductive or esteoconductive materials that may be injected into vertebral endplates include collagen, fibrin, albumin, karatin, silk, elastin, demineralized bone matrix, or particulate bone. Various bone growth promoting biologic materials may also be used including mysenchymal stem cells, hormones, growth factors such as transforming growth factor beta (TGFb) proteins, bone morphogenic proteins (including BMP and BMP2), or platelet derived growth factors. Additional materials that can be injected into vertebral bodies are disclosed in U.S. Pub. No. 2005/0267577, which is hereby incorporated by reference.

Treating Multiple Areas

Treatment, stabilization, and/or reconstruction of the vertebral column may be diagnosed and carried out in a systematic manner depending upon the conditions and material or systems available for treatment. To achieve an improved clinical outcome and a stable result, multiple regions of the vertebral column may be treated.

An objective for treating multiple areas may include one or more of the following benefits: more immediate and adequate stabilization, more accurate anatomical correction, accelerated healing and/or improved clinical outcomes due to mutual reinforcements between the treated areas. The treated regions and employed devices can vary depending upon clinical objectives such as elimination or reduction of motion, restoration or increase of motion, elimination or reduction of intervertebral collapse, restoration or maintenance of disc height, elimination or reduction of hyperlordosis, restoration or increase of lordosis, elimination or reduction of hyperkyphosis, restoration or increase of kyphosis, correction of scoliosis, improvement of spinal alignment in the sagital and/or coronal plane, restoration or increase of vertebral/endplate strength, restoration or increase of vertebral/endplate density, acceleration of intervertebral fusion, and achieving differential stiffness or motion at different regions.

Interbody/Vertebral Body

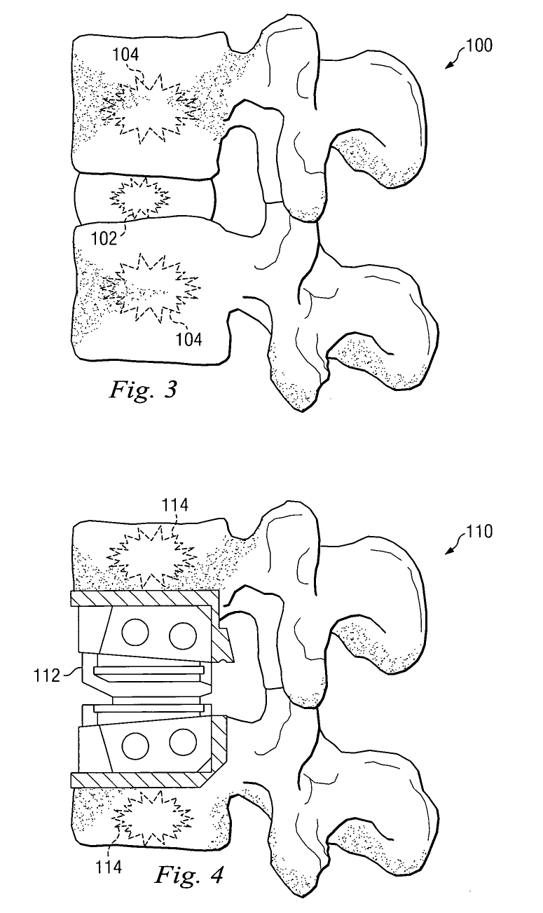

In one example, an intervertebral body system and a vertebral body treatment chosen from the systems described above, may be combined. As shown in FIG. 3, a multiple region system 100 may include an intervertebral treatment 102 such as injectable allogenic collagen with or without cells or growth factors. The system 100 may also include vertebral body augmentation material 104 which may be, for example, PMMA bone cement injected using a vertebroplasty procedure.

In another example, as shown in FIG. 4, a multiple region system 110 may include a motion preserving intervertebral disc prosthesis 112 such as the MAVERICK system offered by or developed by Medtronic, Inc. The system 110 may also include vertebral body augmentation material 114 such as hydroxyapatite-tricalcium phosphate bone cement which may be injected following a vertebral body void creation procedure. The material 114 may reinforce osteoporotic bone adjacent to the treated interbody level.

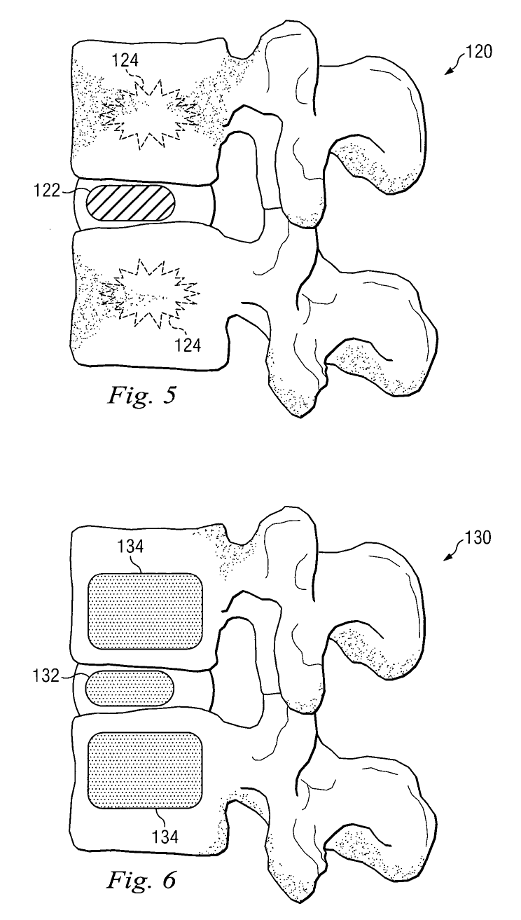

In another example, as shown in FIG. 5, a multiple region system 120 may include a nucleus replacement device 122 such as a NAUTILUS device offered by or developed by Medtronic, Inc. The system 120 may also include vertebral body augmentation material 124 which may be, for example, PMMA bone cement injected using a vertebroplasty procedure.

In another example, as shown in FIG. 6, a multiple region system 130 may include a disc treatment 132 such as an injection with allogenic collagen with or without mysenchymal stem cells or growth factors. The system 130 may also include vertebral body augmentation material 134 which may be, for example, an injectable material such as polyvinyl alcohol (PVA) hydrogel, polyurethane, collagen, demineralized bone matrix, gelatin, polysaccharide, hyaluronic acid, keratin, albumin, silk, elastin, fibrin polymethylmethacrylate (PMMA), calcium phosphate, hyrdroxyapatite-tricalcium phosphate (HA-TCP) compounds, bioactive glasses, polymerizable matrix comprises a bisphenol-A dimethacrylate, or CORTOSS™ by Orthovita of Malvern, Pa. (generically referred to as a thermoset cortical bone void filler) or their combinations.

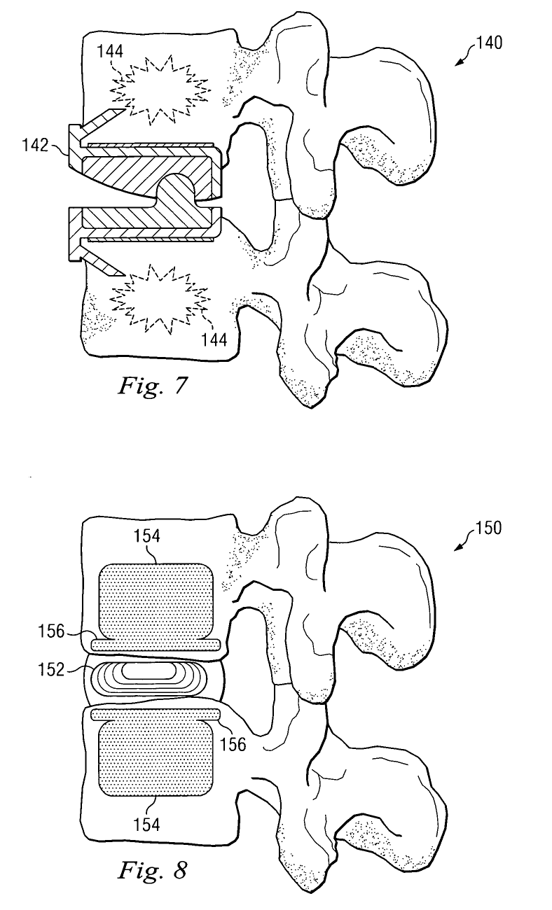

In another example, as shown in FIG. 7, a multiple region system 140 may include a motion preserving intervertebral disc prosthesis 142 such as the PRESTIGE system offered by or developed by Medtronic, Inc. The system 140 may also include vertebral body augmentation material 144 which may be, for example, PMMA bone cement injected following a vertebral body void creation procedure using a balloon such as kyphoplasty. The use of PMMA may allow the bone screws used to anchor the motion preserving disc 142 to attain better purchase within the bone.

It is understood that the combination of treatment methods and devices described in FIGS. 3-7 are merely exemplary and that other materials and systems may be chosen to achieve a desired result involving the intervertebral body and vertebral body regions.

Interbody/Verterbal body/Endplates

In another example, as shown in FIG. 8, a multiple region system 150 may include a nucleus replacement device 152 such as a NAUTILUS device offered by or developed by Medtronic, Inc. The system 150 may also include vertebral body treatment 154 which may be injectable collagen combined with BMP2. The treatment 154 may be useful to strengthen the bony tissue to better support the adjacent restored disc.

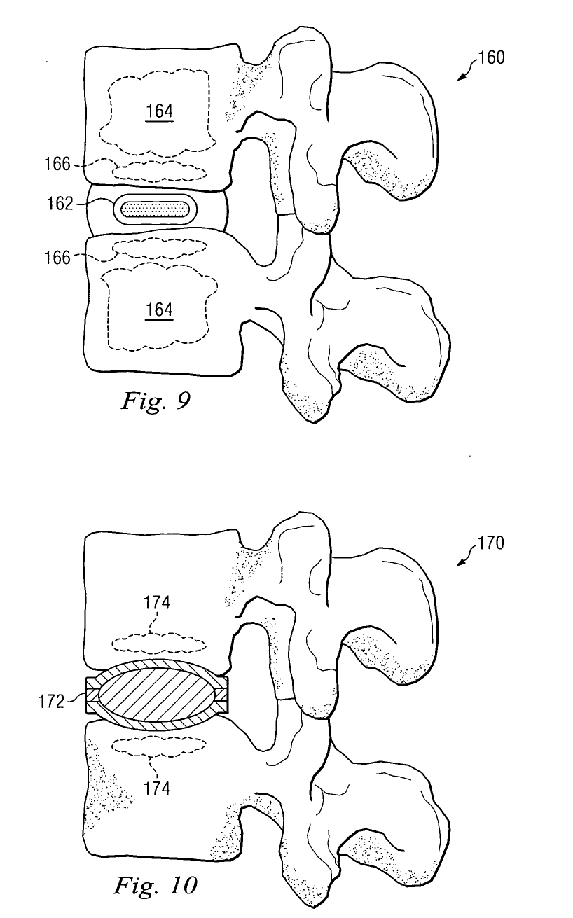

In another example, as shown in FIG. 9, a multiple region system 160 may include a nucleus supplementation system 162 such as injectable polyvinyl alcohol hydrogel. The system 160 may also include vertebral body augmentation material 164 and endplate reinforcement material 166 which may be, for example, PMMA bone cement injected following a bone void creation procedure.

Other examples include, but are not limited to, the following combinations: 1) the NAUTILUS nucleus implant and PMMA bone cement for the vertebral body plus the endplate region, 2) the BRYAN disc prosthesis and HA-TCP for the vertebral body plus the endplate region, 3) the SATELLITE nucleus implant and HA-TCP with BMP2 for the vertebral body plus the endplate region, 4) the MAVERICK disc prosthesis and collagen with BMP2 for the vertebral body plus the endplate region, and 5) the NAUTILUS nucleus implant and collagen with BMP2 and stem cells for the vertebral body plus the endplate region.

It is understood that the combination of treatment methods and devices described in FIGS. 8and 9 are merely exemplary and that other materials and systems may be chosen to achieve a desired result involving the intervertebral body, vertebral body, and endplate regions.

Interbody/Endplate

In another example, as shown in FIG. 10, a multiple region system 170 may include an intervertebral system 172 such as a motion preserving disc having biconcaval endplates between which a core member extends. One example of such a motion preserving disc is the BRYAN disc offered by or developed by Medtronic, Inc. The system 170 may also include endplate augmentation material 174 which may be, for example, PMMA bone cement.

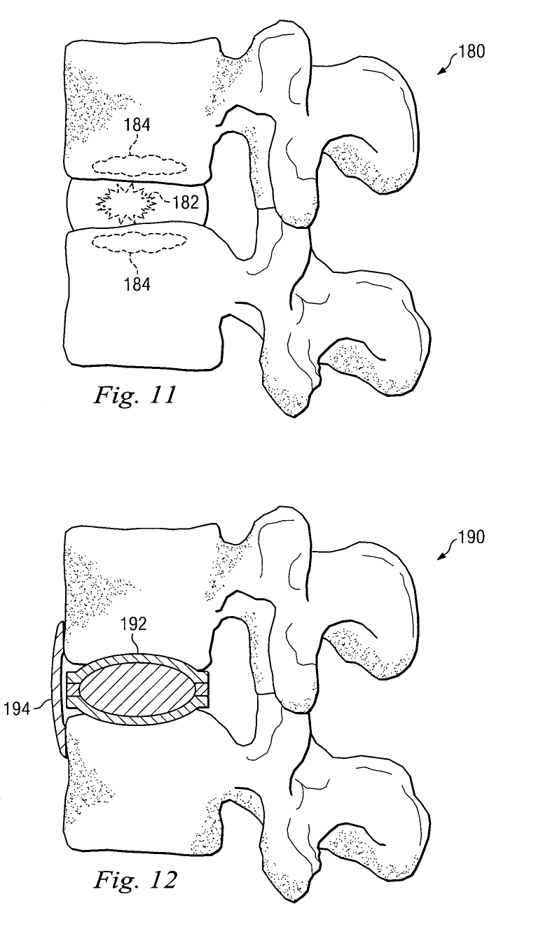

In another example, as shown in FIG. 11, a multiple region system 180 may include an intervertebral treatment 182 such as injectable allogenic collagen with or without cells or growth factors to restore or maintain disc height. This restoration may increase intradiscal pressure and may require endplate region reinforcement. Thus, the system 180 may also include endplate augmentation material 184 which may be, for example, hydroxyapatite-tricalcium phosphate bone cement.

Other examples include but are not limited to the following combinations: 1) the NAUTILUS nucleus implant and PMMA bone cement for the endplate region, 2) the BRYAN disc prosthesis and HA-TCP for the endplate region, 3) the SATELLITE nucleus implant and HA-TCP with BMP2 for the endplate region, 4) the MAVERICK disc prosthesis and collagen with BMP2 for the endplate region, and 5) the NAUTILUS nucleus implant and collagen with BMP2 and stem cells for the endplate region.

It is understood that the combination of treatment methods and devices described in FIGS. 10 and 11 are merely exemplary and that other materials and systems may be chosen to achieve a desired result involving the intervertebral body and endplate regions.

Interbody/Anterior

In another example, as shown in FIG. 12, a multiple region system 190 may include an intervertebral system 192 such as a motion preserving disc having biconcaval endplates between which a core member extends. One example of such a motion preserving disc is the BRYAN disc offered by or developed by Medtronic, Inc. The system 190 may also include an anterior system 194 which may be a flexible plate connected to anterior surfaces of adjacent vertebrae with bone screws.

Other examples include, but are not limited to, the following combinations: 1) the RayMedica's PDN disc nucleus implant and an elastic anterior tension band, 2) the MAVERICK disc prosthesis and a flexible woven anterior plate, 3) injectable collagen for intervertebral disc space and a resorbable polylactide-based anterior plate, 4) the NAUTILUS disc nucleus implant and a flexible anterior band, and 5) LT cages for intervertebral space and an anterior PEEK plate.

It is understood that the combination of treatment methods and devices described in FIG. 12 is merely exemplary and that other materials and systems may be chosen to achieve a desired result involving the interbody and anterior regions.

Interbody/Vertebral Body/Endplate/Anterior

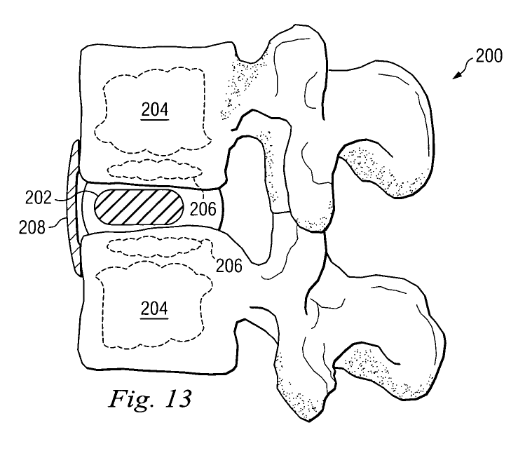

In another example, as shown in FIG. 13, a multiple region system 200 may include a nucleus replacement device 202 such as a NAUTILUS device offered by or developed by Medtronic, Inc. The system 200 may also include vertebral body augmentation material 204 and endplate reinforcement material 206 which may be, for example, PMMA bone cement injected following a bone void creation procedure for the vertebral body. The system 200 may also include an anterior system 208 which may be a flexible anterior plate attached to the anterior faces of adjacent vertebral bodies with bone screws.

Other examples include, but are not limited to, the following combinations: 1) the MAVERICK disc prosthesis, PMMA bone cement for the vertebral body and an elastic anterior tension band, 2) the PRESTIGUE disc prosthesis, HA-TCP for the vertebral body and a flexible woven anterior plate, 3) the LT cage, HA-TCP with BMP2 for the vertebral body plus the endplate region and a resorbable polylactide-based anterior plate, 4) the NAUTILUS nucleus implant, collagen with BMP2 for the endplate region and a flexible anterior band, and 5) the SATELLLITE nucleus implant, collagen with BMP2 and stem cells for the endplate region and an anterior PEEK plate.

It is understood that the combination of treatment methods and devices described in FIG. 13 is merely exemplary and that other materials and systems may be chosen to achieve a desired result involving the interbody, vertebral body, endplate, and anterior regions.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “anterior,” “posterior,” “superior,” “inferior,” “upper,” and “lower” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A method of treating a spinal condition comprising:
placing disc replacement material in an intervertebral disc, thereby altering a loading characteristic of an adjacent vertebrae; and
supporting the adjacent vertebrae by applying a bone filling material to either a superior or inferior vertebral body adjacent to the intervertebral disc.

2. The method of claim 1 wherein the disc placement material is configured to augment the intervertebral disc.

3. The method of claim 1 wherein the disc replacement material is a fusion device.

4. The method of claim 3 wherein the fusion device is a cage.

5. The method of claim 3 wherein the fusion device is a spacer.

6. The method of claim 1 wherein the disc replacement material is a motion preservation device comprising a curved protrusion adapted to articulate with a curved recess.

7. The method of claim 1 wherein the disc replacement material is a motion preservation device comprising a central component adapted to articulate between a pair of endplate components.

8. The method of claim 1 wherein the disc replacement material comprises an elastomeric material adapted to occupy an area within a natural annulus.

9. The method of claim 1 wherein the step of placing replacement material in the intervertebral disc comprises injecting material into a natural nucleus of the intervertebral disc.

10. The method of claim 8 wherein the injected material is curable in situ.

11. The method of claim 1 wherein applying a bone filling material further comprises expanding a void creation device within the superior or inferior vertebral body to create a void and filling at least a portion of the created void with a hardenable material.

12. The method of claim 10 wherein the void creation device is an inflatable device.

13. The method of claim 1 wherein the bone filling material includes a material selected from a group consisting of: polymethylmethacrylate (PMMA), calcium phosphate, hyrdroxyapatite-tricalcium phosphate (HA-TCP) compounds, bioactive glasses, polymerizable matrix comprises a bisphenol-A dimethacrylate, and thermoset cortical bone void filler.

14. The method of claim 1 wherein the bone filling material includes a material selected from a group consisting of: demineralized bone matrix, collagen, gelatin, polysaccharide, hyaluronic acid, keratin, albumin, fibrin, and combinations thereof.

15. The method of claim 1 wherein the bone filling material includes inorganic particles selected from a group consisting of: hydroxyapatite, fluorapatite, oxyapatite, Wollastonite, anorthite, calcium fluoride, agrellite, devitrite, canasite, phlogopite, monetite, brushite, octocalcium phosphate, Whitlockite, tetracalcium phosphate, cordierite, Berlinite, and mixtures thereof.

16. The method of claim 1 wherein the bone filling material comprises growth factors, hormones, or cells

17. The method of claim 15 wherein the bone growth factor includes an element selected from a group consisting of: BMP, TGF, and platelet-derived material.

18. The method of claim 1 wherein the bone filling material comprises mysenchymal stem cells.

19. A method of treating a spinal condition comprising:
placing an augmentation material in an intervertebral disc, thereby altering a characteristic of a first vertebrae; and
supporting the adjacent vertebrae by replacing at least a portion of a second, adjacent vertebral with an artificial vertebral body.

20. The method of claim 19 wherein the augmentation material includes a nucleus replacement implant.

21. A method of treating a spinal condition comprising:
placing a disc replacement system in an intervertebral disc space between a pair of vertebrae; and
supporting the disc replacement system to maintain disc height by connecting an anterior system between the pair of vertebrae.

22. The method of claim 21 wherein the anterior system comprises a PEEK plate.

23. The method of claim 21 wherein the anterior system comprises a bioresorbable plate.

24. The method of claim 21 wherein the anterior system comprises a woven or braided structure.

25. The method of claim 21 wherein the anterior system comprises an elastic tension band.

26. A method of treating a spinal condition comprising:
placing a disc replacement system in an intervertebral disc space;
supporting the disc replacement system to maintain disc height by connecting an anterior system between a pair of vertebrae; and
reinforcing at least one of the pair of vertebrae to support the disc replacement system by treating at least one of the pair of vertebrae with a vertebral body treatment material.

27. The method of claim 26 further comprising:
reinforcing an endplate of at least one of the pair of vertebrae to support the disc replacement system by treating the end plate with an endplate treatment material.

28. The method of claim 26 wherein the vertebral body treatment material comprises polymethylmethacrylate.

29. The method of claim 26 wherein the vertebral body treatment material includes a material selected from a group consisting of: hydroxyapatite, tricalcium phosphate, and injectable collagen.

30. The method of claim 27 wherein the endplate treatment material comprises BMP.

31. The method of claim 27 wherein the endplate treatment material comprises polymethylmethacrylate.

32. The method of claim 26 wherein the anterior system includes an element selected from a group consisting of: a PEEK plate, a bioresorbable plate, a woven or braided structure, and an elastic tension band.

33. A method of treating a spinal condition comprising:
placing a disc replacement system in an intervertebral disc space between a pair of vertebrae; and
reinforcing an endplate of at least one of the pair of vertebrae to support the disc replacement system by treating the endplate with an endplate treatment material.

34. The method of claim 33 wherein the disc replacement system is a motion preservation system.

35. The method of claim 34 wherein the motion preservation system comprises a single pair of articulating surfaces.

36. The method of claim 34 wherein the motion preservation system comprises two pairs of articulating surfaces.

37. The method of claim 33 wherein the endplate treatment material comprises hydroxyapatite.

38. The method of claim 33 wherein the endplate treatment material comprises tricalcium phosphate.

39. The method of claim 33 wherein the endplate treatment material comprises a bone growth factor.

40. The method of claim 33 wherein the endplate treatment material comprises collagen.

41. A method of treating a spinal condition comprising:
placing a disc replacement system in an intervertebral disc space between a pair of vertebrae; and
reinforcing an endplate of at least one of the pair of vertebrae to support the disc replacement system by treating the endplate with an endplate treatment material.

42. The method of claim 41 further comprising:
reinforcing at least one of the pair of vertebrae to support the disc replacement system by treating at least one of the pair of vertebrae with a vertebral body treatment material; and

43. The method of claim 41 wherein the endplate treatment material includes a material selected from a group consisting of: polymethylmethacrylate, DBM, platelet-derived growth factors, and calcium sulfate.

44. The method of claim 42 wherein the vertebral body treatment material includes a material selected from a group consisting of: polymethylmethacrylate, DBM, platelet-derived growth factors, and calcium sulfate.

45. The method of claim 41 wherein the disc replacement system includes an element from a group consisting of: an elastomeric material, a mechanical spring element, an elastomeric nucleus replacement implant, and an injectable polymer.

46. The method of claim 41 wherein the disc replacement system comprises an intervertebral portion and a bridge portion extending posteriorly from the intervertebral portion.
